(19) United States
(12) Patent Application Publication
(10) Pub. No.: US 2024/0349984 A1
(43) Pub. Date: Oct. 24, 2024

(54) SYSTEMS AND METHODS FOR GENERATING IMAGES OF A SELECTED IMAGING PLANE USING A FORWARD-FACING IMAGING ARRAY

(71) Applicant: INTUITIVE SURGICAL OPERATIONS, INC., Sunnyvale, CA (US)

(72) Inventors: Serena H. Wong, Los Altos, CA (US); Samuel Raybin, San Jose, CA (US)

(21) Appl. No.: 18/641,035

(22) Filed: Apr. 19, 2024

Related U.S. Application Data
(60) Provisional application No. 63/497,603, filed on Apr. 21, 2023.

(51) Int. Cl. A61B 1/00

(52) U.S. Cl. CPC A61B 1/000094; A61B 1/00006; A61B 1/00013; A61B 1/0008

(57) ABSTRACT

A system may comprise an elongate flexible instrument including an imaging device disposed at a distal end portion of the elongate flexible instrument. The imaging device may include a multi-directional imaging array. The elongate flexible instrument may also include a localization sensor extending within the elongate flexible instrument. The system may also comprise a controller comprising one or more processors configured to register the localization sensor to a patient anatomy and receive orientation data for the distal end portion of the elongate flexible instrument from the localization sensor. Based on the orientation data, an imaging plane of the imaging device may be selected. An image in the selected imaging plane may be displayed. The image may be generated by imaging data from the multi-directional imaging array of the imaging device.

### CROSS-REFERENCED APPLICATIONS

This application claims priority to and benefit of U.S. Provisional Applications No. 63/497,603 filed Apr. 21, 2023 and entitled “Systems and Methods for Generating Images of a Selected Imaging Plane Using a Forward-Facing Imaging Array,” which is incorporated by reference herein in its entirety.

### FIELD

The present disclosure is directed to systems and methods for generating images having imaging planes of a selectable orientation, using a forward-facing imaging array.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation and deployment of medical tools may be assisted using images of the anatomic passageways and surrounding anatomy, obtained intra-operatively. Intra-operative imaging alone or in combination with pre-operative imaging may provide improved navigational guidance and confirmation of engagement of an interventional tool with the target tissue. Improved systems and methods are needed for providing image guidance while minimizing the size of the medical tool.

### SUMMARY

Consistent with some examples, a system may comprise an elongate flexible instrument including an imaging device disposed at a distal end portion of the elongate flexible instrument. The imaging device may include a multi-directional imaging array. The elongate flexible instrument may also include a localization sensor extending within the elongate flexible instrument. The system may also comprise a controller comprising one or more processors configured to register the localization sensor to a patient anatomy and receive orientation data for the distal end portion of the elongate flexible instrument from the localization sensor. Based on the orientation data, an imaging plane of the imaging device may be selected. An image in the selected imaging plane may be displayed. The image may be generated by imaging data from the multi-directional imaging array of the imaging device.

Consistent with some examples, a method may comprise registering a localization sensor to a patient anatomy, the localization sensor extending within an elongate flexible instrument and receiving orientation data for a distal end portion of the elongate flexible instrument from the localization sensor. Based on the orientation data, an imaging plane of an imaging device disposed at a distal end of the elongate flexible instrument may be selected. An image in the selected imaging plane may be displayed. The image may be generated by imaging data from a multi-directional imaging array of the imaging device.

Other embodiments include corresponding computer systems, apparatus, and computer programs recorded on one or more computer storage devices, each configured to perform the actions of the methods.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A illustrates an example of a medical instrument system in a patient anatomy near a target tissue, according to some examples.

FIG. 1B illustrates a guidance tool for display during a medical procedure, according to some examples.

FIG. 1C illustrates a guidance tool for display during a medical procedure, according to some examples.

FIG. 1D illustrates a guidance tool for display during a medical procedure, according to some examples.

FIG. 2A is a side view of a medical instrument within an anatomic passageway, according to some examples.

FIG. 2B is a distal end view of the medical instrument of FIG. 2A, according to some examples.

FIG. 2C is an image generated by the medical instrument of FIG. 2A, according to some examples.

FIG. 2D is a side view of the medical instrument of FIG. 2A at a different roll angle within an anatomic passageway, according to some examples.

FIG. 2E is a distal end view of the medical instrument of FIG. 2D, according to some examples.

FIG. 2F is an image generated by the medical instrument of FIG. 2D, according to some examples.

FIG. 3 illustrates an example of a medical instrument system in a patient anatomy near a target tissue, according to some examples.

FIG. 4A is a side view of a medical instrument within an anatomic passageway, according to some examples.

FIG. 4B is a distal end view of the medical instrument of FIG. 4A, according to some examples.

FIG. 4C is an image generated by the medical instrument of FIG. 4A, according to some examples.

FIG. 4D is a side view of the medical instrument of FIG. 4A at a different roll angle within an anatomic passageway, according to some examples.

FIG. 4E is a distal end view of the medical instrument of FIG. 4D, according to some examples.

FIG. 4F is an image generated by the medical instrument of FIG. 4D, according to some examples.

FIG. 4G is a side view of the medical instrument of FIG. 4A at a different roll angle within an anatomic passageway, according to some examples.

FIG. 4H is a distal end view of the medical instrument of FIG. 4G, according to some examples.

FIG. 4I is an image generated by the medical instrument of FIG. 4G, according to some examples.

FIG. 5A is a side view of a medical instrument within an anatomic passageway, according to some examples.

FIG. 5B is a distal end view of the medical instrument of FIG. 5A, according to some examples.

FIG. 5C is an image generated by the medical instrument of FIG. 5A, according to some examples.

FIG. 5D is a side view of the medical instrument of FIG. 5A at a different roll angle within an anatomic passageway, according to some examples.

FIG. 5E is a distal end view of the medical instrument of FIG. 5D, according to some examples.

FIG. 5F is an image generated by the medical instrument of FIG. 5D, according to some examples.

FIG. 6 is a flowchart illustrating a method for generating an image in a selected visualization plane relative to the anatomic passageway, according to some examples.

FIG. 7A is a flowchart illustrating a method for selecting an image plane based on sensor data, according to some examples.

FIG. 7B is a flowchart illustrating a method for selecting an image plane based on sensor data, according to some examples.

FIG. 8 is a robot-assisted medical system, according to some examples.

FIGS. 9A and 9B are simplified diagrams of a medical instrument system according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The techniques disclosed in this document may be used to enhance intra-operative imaging instruments and their use in minimally invasive procedures. In some examples, intra-operative imaging data may be utilized to verify real-time accurate placement of a treatment or diagnostic tool within an anatomical target during a medical procedure. For example, an imaging instrument may be used to provide direct visual guidance of a target tissue and surrounding vulnerable tissue in preparation for and during a procedure to advance an interventional tool toward the target tissue. In various examples, the imaging instrument may include a forward-facing imaging array and a localization sensor that allows a selected image plane of the imaging array data to be displayed. Although some of the imaging instruments described herein are ultrasound imaging instruments, it is contemplated that the systems and methods described herein may be applied to other imaging and sensing modalities without departing from the scope of the present disclosure.

The systems and techniques described in this document may be used in a variety of medical procedures that may improve accuracy and outcomes through use of intra-operative imaging. For example, intra-operative imaging may be used to biopsy lesions or other tissue to, for example, evaluate the presence or extent of diseases such as cancer or surveil transplanted organs. As another example, intra-operative imaging may be used in cancer staging to determine via biopsy whether the disease has spread to lymph nodes. The medical procedure may be performed using hand-held or otherwise manually controlled imaging probes and tools (e.g., a bronchoscope). In other examples, the described imaging probes and tools many be manipulated with a robot-assisted medical system.

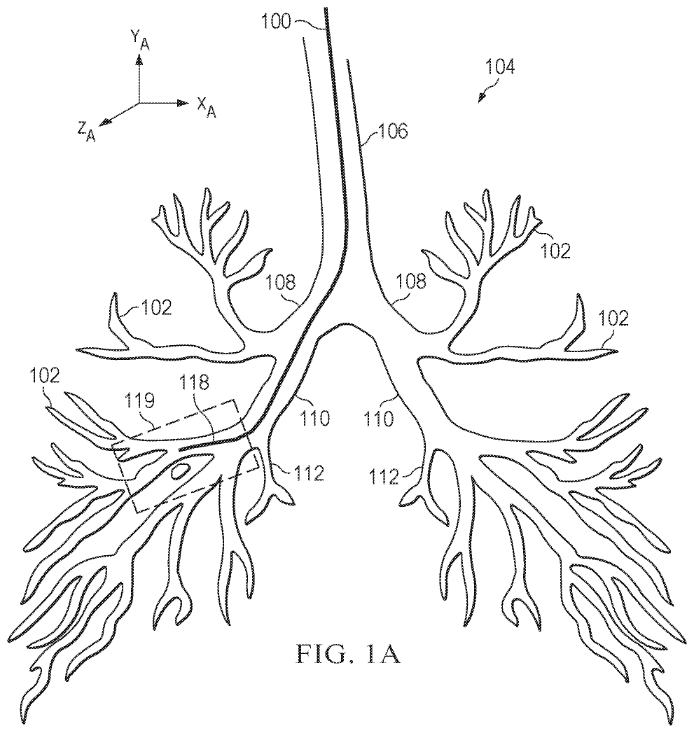

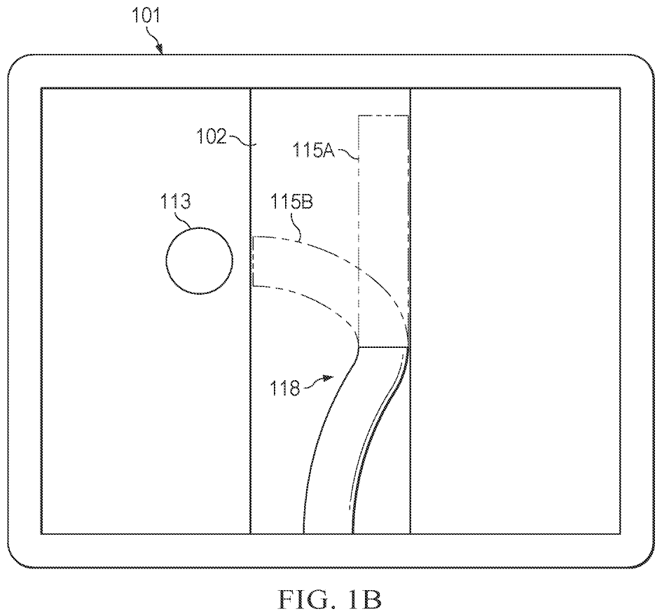

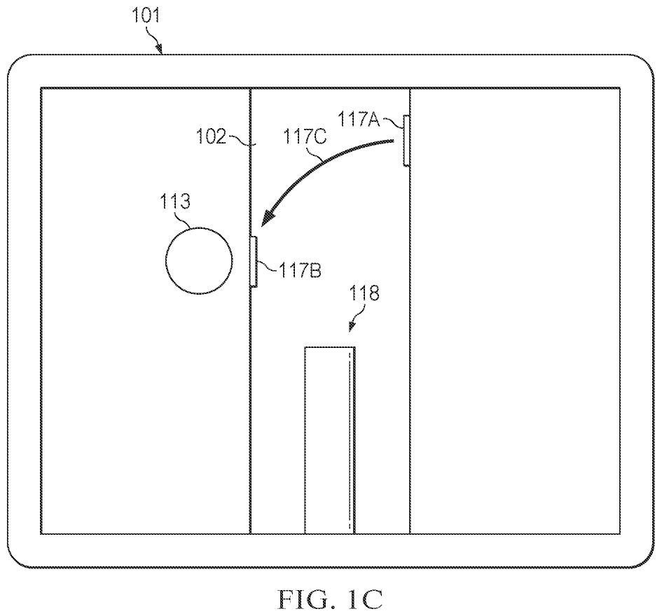

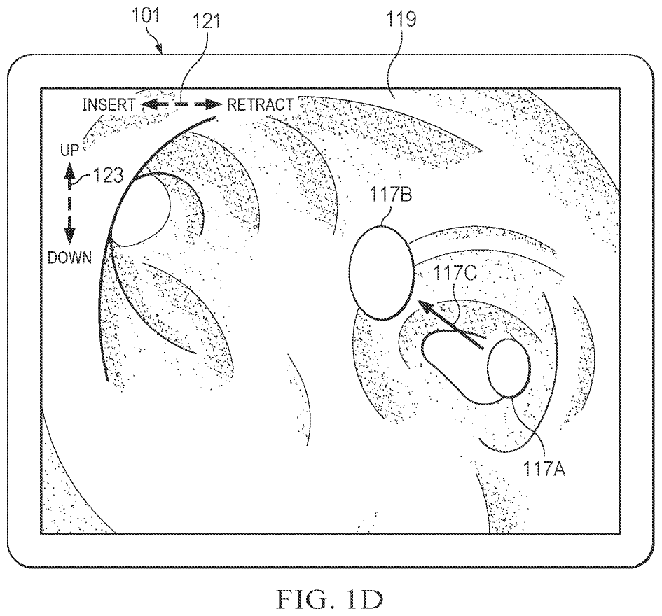

FIG. 1A illustrates an elongated medical instrument system 100 extending within branched anatomic passageways or airways 102 of an anatomic structure 104. In some examples the anatomic structure 104 may be a lung and the passageways 102 may include the trachea 105, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument system 100 may be advanced into an anatomic opening (e.g., a patient mouth) and through the anatomic passageways 102 to perform a medical procedure, such as a biopsy, at or near a target tissue 113 in an anatomic region 119.

When performing a medical procedure, such as a lung biopsy, a clinician may sample target tissue to determine characteristics of the target. For some biopsy procedures, side-facing curvilinear ultrasound imaging arrays positioned at a distal end of a flexible device may be used. A side-facing array may produce an image of an anatomy sector along a plane parallel to the longitudinal axis of the passageway (and, generally, the longitudinal axis of the flexible device shaft). Regardless of the rotational orientation of the device (due to the side-facing nature of the imaging array) the image displayed to the clinician may be in a plane parallel to the longitudinal axis of the airway. As such, a clinician may be accustomed to, and may prefer, viewing the target in an imaging plane that is parallel to the longitudinal axis of the airway. For some procedures, the use of a forward-facing ultrasound array (e.g., exposed on a distal face of the elongate flexible device) may be preferable to a side-facing array. For example, an ultrasound instrument with a forward-facing array may have a smaller outer diameter, allowing the instrument to extend into smaller, more distal airways and allowing for more flexibility and maneuverability. The forward-facing array may also be useful if navigational control is provided by a robotic-assistance that does not include control of an axial rotation degree of freedom of the instrument. Some clinicians may find navigation of an ultrasound instrument with a forward-facing array to be more intuitive. As compared to a side-facing ultrasound transducer that may have a relatively long, rigid distal end portion, a forward-facing array may have a shorter rigid distal end portion that may require less force to control steering, navigation, and apposition.

To capture an image of the target tissue and nearby vulnerable anatomy external to an anatomic passageway, the elongate flexible device may be bent to face the wall of the anatomic passageway. Depending on the direction of the bend, a linearly-arranged, forward-facing array may generate sector images in a variety of image planes that range from parallel to the longitudinal axis of the airway to perpendicular to the longitudinal axis of the airway. In some examples, guidance may be provided to a clinician to assist with positioning the elongate flexible device. FIG. 1B illustrates a graphical user interface 101 that may be displayed (e.g. on a display system 510) during a medical procedure to provide guidance in positioning the distal end portion 118 of the medical instrument system 100 within a passageway 102. In this example, a pre-operative model (e.g., a pre-operative CT model) of the anatomic structure 104 may be registered to the medical instrument system 100 frame of reference. The graphical user interface 101 may include a synthetic image of the current position of the distal end portion 118 with reference to the passageway 102 and the target tissue 113 (as provided by the registered pre-operative model). A guidance marker 115A may guide the user to extend the distal end portion 118 a distance beyond the target tissue 113. After the distal end portion 118 is extended based on the guidance 115A, a guidance marker 115B may guide the user to form optimal bend configuration for imaging the target tissue 113. In various examples, the guidance markers may be illustrated as synthetic extensions of the current position of the distal end portion 118 or as way point markers that illustrate a guided path in such views as a global three-dimensional view. In some examples, portions of the passageway may be marked with markers, directional indicators, or other textual or graphical guidance. In some examples the guidance may be color coded to provide guidance for a sequence of steps. The graphical guidance may be displayed on a global three-dimensional view or on a synthetic anatomic view. FIG. 1C illustrates the graphical user interface 101 illustrating a global three-dimensional view. In this example, the passageway 102 is marked with an extension marker 117A that indicates the side of the passageway and the extension distance to which the distal end portion 118 should be driven and with an apposition marker 117B which indicates the direction the distal end portion 118 should be facing to access the target 113. In some examples, the extension marker may be rendered with a green color and the destination marker may be rendered with a blue color, but various color choices may be suitable. A marker 117C may be an arrow indicating the direction of bend for the distal end portion 118. FIG. 1D illustrates the graphical user interface 101 illustrating a synthetic anatomic view of the passageway 102. In this example, the extension marker 117A indicates the side of the passageway 102 and the extension distance to which the distal end portion 118 should be driven. The apposition marker 117B indicates the direction the distal end portion 118 should be facing to access the target 113. In some examples the extension marker may be rendered with a green color and the destination marker may be rendered with a blue color, but various color choices may be suitable. The arrow marker 117C may indicate the direction of bend for the distal end portion 118. In some examples, input device guidance 121, 123 may be displayed on the graphical user interface 101 with respect to the anatomic region 119. For example, the guidance 121 may include left and right arrows indicating insertion and retraction direction of a first input device, such as a scroll wheel, of the master assembly (e.g. master assembly 506). The guidance 123 may include up and down arrows indicating up and down motion of a second input device, such as a trackball, of the master assembly.

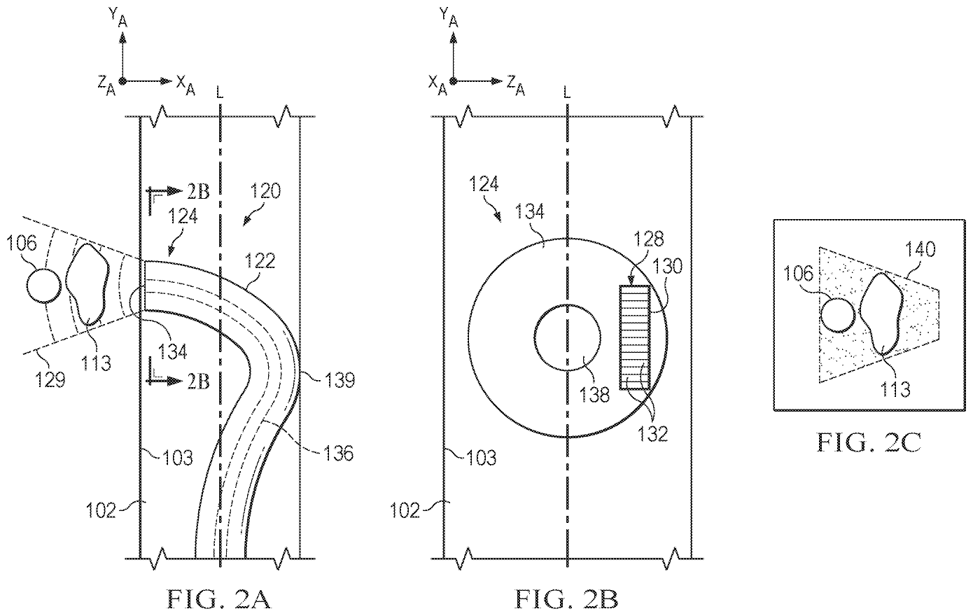

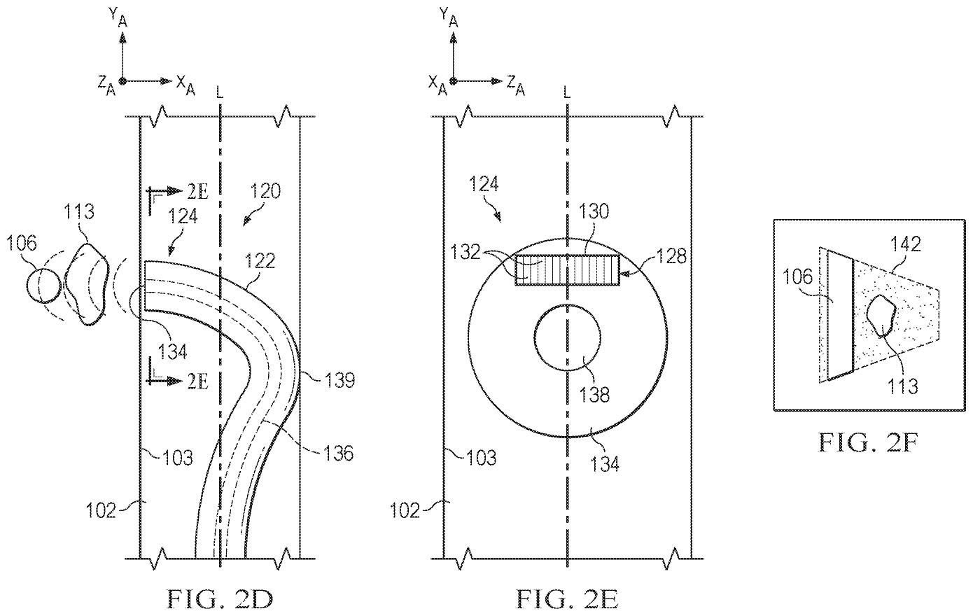

As shown in FIGS. 2A and 2B, an elongated medical instrument system 120 (e.g., the elongated medical instrument system 100) may include an elongate flexible instrument 122. A distal end portion 124 of the elongate flexible instrument 122 may include an imaging device 128, such as an ultrasound imaging device, with an imaging field of view 129. The instrument 122 may be positioned within the anatomic region 119 in a passageway 102 near the target tissue 113. More specifically, a distal face 134 of the instrument 122 may be generally parallel to and in contact with or near a wall 103 of the passageway 102, in the proximity of the target tissue 113. In some examples, a more proximal portion 139 of the instrument may contact a portion of the wall 103, opposite the target tissue 113 to provide a contact force between the distal face 134 and the wall 103 near the target tissue 113. Sensitive or vulnerable anatomic structures 106 (e.g., major blood vessels, lung pleura, large bullae) may be in the vicinity of the target tissue 113, and the medical procedure may be planned and/or monitored to avoid engaging or damaging such structures.

In this example, the ultrasound imaging device 128 may include a forward-facing transducer array 130 including a plurality of linearly aligned transducer elements 132 at the distal end portion 124 of the elongate flexible instrument 122. The forward-facing transducer array 130 may be arranged to image in an antegrade or forward-looking direction. Thus, the imaging field of view 129 of the forward-facing transducer array 130 may extend distally of a distal face 134 of the instrument 122. A channel 136 extending through the elongate flexible instrument 122 may have a distal opening 138 at the distal face 134. With the transducer array 130 aligned generally parallel to a passageway wall 103, an ultrasound image 140 of the target tissue 113 and the nearby anatomic structures 106 may be generated, as shown in FIG. 2C. In this example, the ultrasound image 140 may be a sector image in a visualization plane generally parallel to a longitudinal axis L of the passageway 102 (which may also be parallel to the passageway wall 103).

For some instruments, it may be difficult to control axial rotation and, consequently, the alignment of the transducer array to the longitudinal axis L or the direction of the passageway wall. As a result, the same forward-facing array may generate sector images in a variety of image planes that range from parallel to the longitudinal axis of the airway to perpendicular to the longitudinal axis of the airway. For example, as shown in FIGS. 2D and 2E, if the distal end portion 124 is axially rotated (e.g., rolled about a X_{A} axis) approximately ninety degrees, relative to the arrangement in FIGS. 2A and 2B, the transducer array 130 becomes rotated to extend approximately perpendicular to the direction of the passageway wall 103 and generally perpendicular to the axis longitudinal L. With this axial rotation, an ultrasound image 142 of the target tissue 113 and the nearby anatomic structures 106 may be generated, as shown in FIG. 2F. In this example, the ultrasound image 142 may be a sector image in a visualization plane generally perpendicular to the longitudinal axis A of the passageway 102 (which may also be perpendicular to the passageway wall 103).

As shown in FIGS. 2C and 2F, depending on the direction of the bend, a linearly-arranged, forward-facing array may generate sector images in a variety of image planes that range from parallel to the longitudinal axis of the airway to perpendicular to the longitudinal axis of the airway. Any variety or non-uniformity in the direction of the image of the field of view may create confusion or disorientation for a clinician or another user of the images such as an automated image processing system. Systems and methods that generate images in a selected, predetermined, or otherwise known image plane relative to the anatomic passageway, regardless of the direction of the instrument bend or axial rotation, may provide a more consistent, less confusing display of the patient anatomy to the clinician. For example, the generated image may be selected to be in a plane parallel to the longitudinal axis of the airway, regardless of the direction of the instrument bend. Presenting clinicians with images in imaging planes that have an expected orientation may improve confidence and precision in biopsy or other interventional procedures.

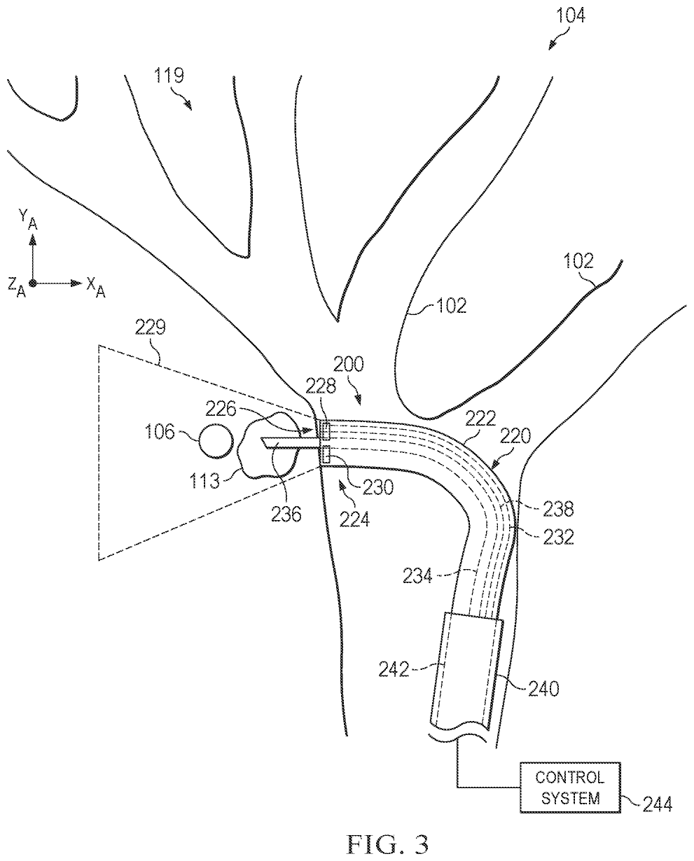

FIG. 3 illustrates an elongated medical instrument system 200 (e.g., the elongated medical instrument system 100) extending within branched anatomic passageways or airways 102 of an anatomical structure 104.

The elongated medical instrument system 200 may include an elongate flexible instrument 220 including a flexible body 222. A distal end portion 224 of the elongate flexible instrument 220 may include an imaging system 226. The imaging system 226 may include an imaging device 228, such as an ultrasound imaging device, that has an imaging field of view 229. In some examples, the imaging system 226 may also include an optical imaging device 230, such as visible light camera and/or a near infrared camera. The elongate flexible instrument 220 may include a localization sensor 232 configured to measure pose information for the distal end portion 224 of the elongate flexible instrument 220. In some examples, the localization sensor 232 may be a six degree of freedom sensor, such as an optical fiber shape sensor, that provides pose (e.g. position and shape data) along at least a portion of the flexible instrument 220. Instead of (or in addition to) an optical fiber shape sensor, the localization sensor 232 may be an electromagnetic (EM) sensor or a plurality of EM sensors positioned at known locations relative to the distal end portion 224 to track the position and orientation of distal end portion 224. A channel 234 may extend through the flexible body 222 and provide passage for an interventional tool 236. The interventional tool 236 may include, for example, a biopsy or tissue sampling tool (e.g., needle or forceps), an ablation tool including a heated or cryo-probe, an electroporation tool, a medication delivery device, a fiducial delivery device, or another type of diagnostic or therapeutic device. In some examples, the interventional tool may be used to deliver a device into or near the target tissue. For example, a radiopaque marker or a drug delivery implant may be delivered by the interventional tool. The elongate flexible instrument 220 may also include a steering system 238 for steering the distal end portion 224 in one or more degrees of freedom. The steering system 238 may include, for example, pull wires, tendons, Bowden cables, or other elongated control mechanisms configured to bend the distal end portion 224. The steering system 238 may be controlled by a robot-assisted manipulator (e.g., manipulator 502) or may be manually manipulated. Optionally, the steering system may be omitted and the instrument 220 may be steered by a robot-assisted delivery catheter. The medical system 200 may also include a control system 244 that may receive information from and provide instructions to the imaging system 226, the localization sensor 232, the interventional tool 236, and/or the steering system 238. In some examples, the control system may include or be included in a robot-assisted medical system control system (e.g. control system 912).

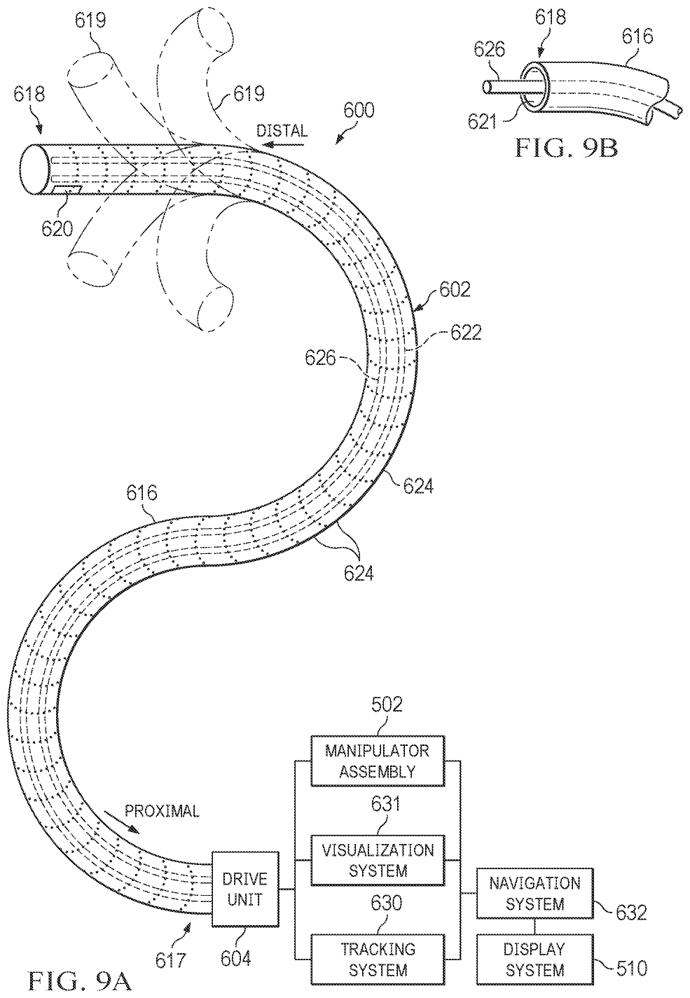

In some examples, the medical system 200 may include a delivery catheter 240 with a channel 242 through which the elongate flexible instrument 220 may be delivered into the anatomic passageway 102. For example, the elongate flexible instrument 220 may be slidably advanced or retracted within channel 242. In some examples, the delivery catheter may be a manually controlled bronchoscope or a robot-assisted steerable catheter system. An example of a robot-assisted delivery catheter system that is bendable and steerable in multiple degrees of freedom is described below in FIGS. 9A and 9B (e.g. the system 900). In some examples, the delivery catheter may be omitted and the elongate flexible instrument 220 may be inserted directly into the patient anatomy, without the path guidance or external steering systems of a delivery catheter. In some examples, the elongate flexible instrument 220 may be integrated with the components of the delivery catheter (e.g., the system 900) such that the components of the elongated flexible instrument 220 remain fixed relative to a distal end of the delivery catheter.

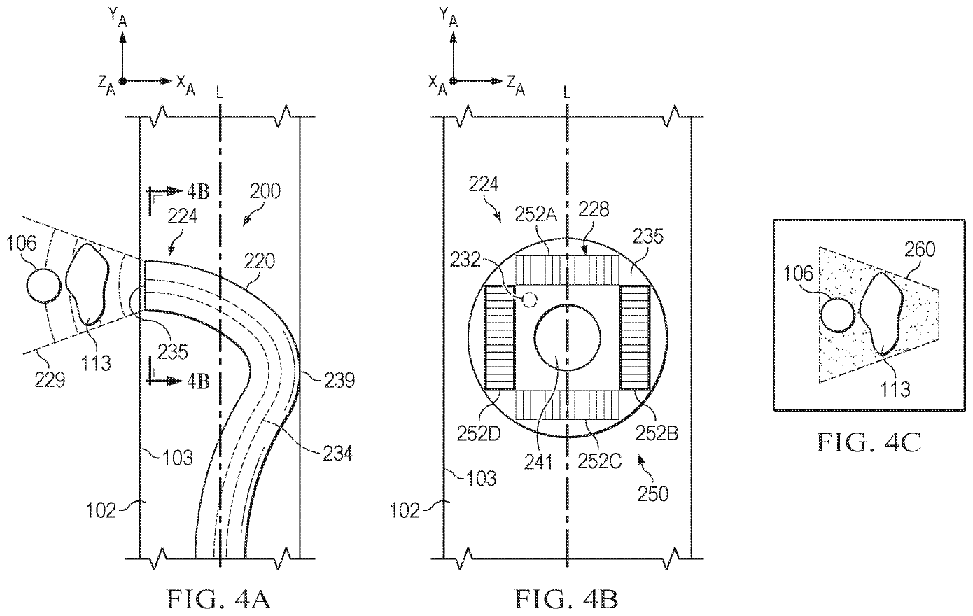

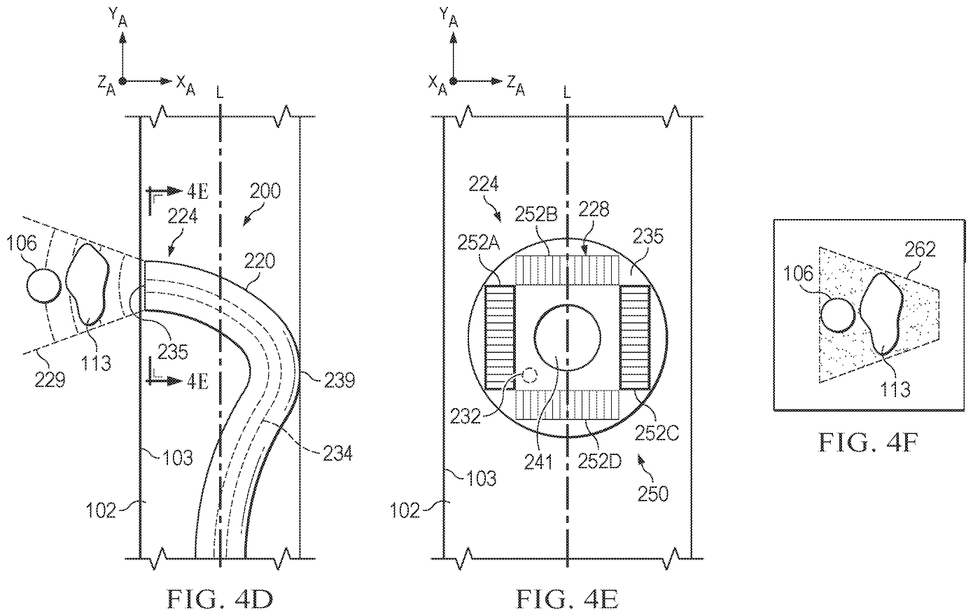

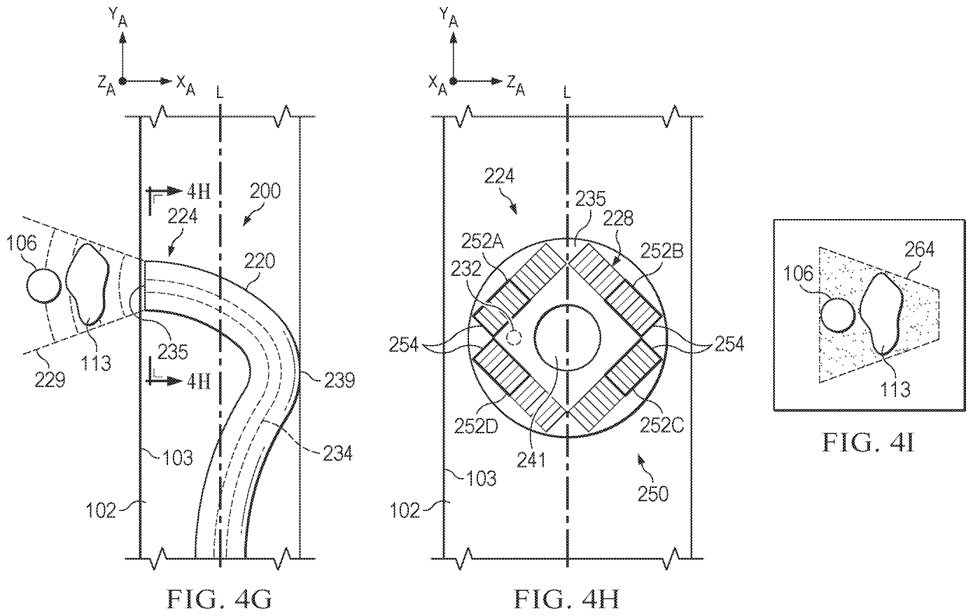

As shown in FIGS. 4A and 4B, the instrument 220 may be positioned in the passageway 102 near the target tissue 113. More specifically, a distal face 235 of the instrument 220 may be generally parallel to and in contact with or near a wall 103 of the passageway 102, in the proximity of the target tissue 113. In some examples, a more proximal portion 239 of the instrument may contact a portion of the wall 103, opposite the target tissue 113 to provide a contact force between the distal face 235 and the wall 103 near the target tissue 113. The channel 234 extending through the elongate flexible instrument 220 may have a distal aperture or opening 241 at the distal face 235.

In this example, the imaging device 228 may include a forward-facing, multi-directional, ultrasound transducer array 250 including an array or set 252A of linearly aligned transducer elements, a set 252B of linearly aligned transducer elements, a set 252C of linearly aligned transducer elements, and a set 252D of linearly aligned transducer elements. In some examples, each of the transducer sets 252A-D may be similar to the transducer array 130, including linearly aligned transducer elements 132. In this example, the set 252A and set 252C may extend generally parallel to each other, each on an opposite side of distal opening 241 or on opposite sides of a central axis of the instrument. The sets 252B and 252D may extend generally parallel to each other, each on an opposite side of distal opening 241. The sets 252B and 252D may extend generally orthogonal to the sets 252A and 252C. The forward-facing transducer array 250 may be arranged to image in an antegrade or forward-looking direction. Thus, the imaging field of view 229 of the forward-facing transducer array 250 may extend distally of a distal face 235 of the instrument 220. With the transducer sets arranged in multiple and different linear directions, specific transducer elements or specific sets of transducer elements may be used to capture an image in a preferred imaging plane (e.g. an imaging plane which is parallel to the longitudinal axis of the passageway), regardless of the direction the distal end portion of the imaging device is bent or the axial rotation of the imaging device. In other examples, the transducer sets may have other multi-directional configurations including angled or otherwise non-orthogonal linear or non-linear sets of transducer elements.

In this example, the localization sensor 232 may be a shape sensor terminating near the transducer sets 252A and 252D. In other examples, the localization sensor may terminate in other known or predetermined positions and orientations relative to the multi-directional array. In other examples, the localization sensor may include a plurality of electromagnetic sensors that together provide six degree of information shape information for the instrument.

During a procedure, the localization sensor 232 may be registered to the patient anatomy and to a pre-operative model of the anatomic structure 104. With the distal face 235 bent into contact with or close proximity to the passageway wall 103, the transducer array 250 may be aligned generally parallel to the passageway wall 103. Data received from the localization sensor 232 may provide pose information for the distal face 235 and distal end portion 224 of the instrument 220. Because the localization sensor 232 has a known position and orientation relative to the transducer array 250, the orientation of the transducer array 250 may be determined relative to the registered pre-operative model. Thus, the orientation of the transducer array 250 relative to a central axis or wall of the anatomic passageway may be determined. Based on a desired visualization plane (e.g. parallel to the axis of the axis of the passageway or parallel to the wall of the passageway), selected portions of the transducer array 250 may be used to generate an ultrasound image in the desired visualization plane. For example, based on the pose data from the localization sensor 232, the transducer elements of the sets 252B and 252D, parallel to the longitudinal axis L, are determined to be in an orientation to generate an image with a visualization plane parallel to the axis L. Ultrasound image data from the transducer elements of selected sets 252B and/or 252D may be used to generate the ultrasound image 260 of the target tissue 113 and the nearby anatomic structures 106, as shown in FIG. 4C. In this example, the ultrasound image 260 may be a sector image in a visualization plane generally parallel to the longitudinal axis L of the passageway 102 (which may also be parallel to the passageway wall 103).

For some instruments or procedures, it may be difficult to precisely control axial rotation (e.g., roll angle about the axis of the instrument) and, consequently, the alignment of the transducer array 250 to the longitudinal axis L or the direction of the passageway wall. The bend angle and axial orientation of the instrument 220 may cause the distal end portion 124 to engage the passageway wall 103 at any of a variety of orientations. For example, as shown in FIGS. 4D and 4E, the distal face 235 may have an axial rotation (e.g., roll about a X_{A} axis) approximately ninety degrees counter-clockwise, relative to the arrangement in FIGS. 4A and 4B. The transducer array 250 and the localization sensor 232 are also rotated approximately ninety degrees counter-clockwise. With this rotation, an image generated using the same transducer sets 252B, 252D as used to generate image 260 of FIG. 4C would be in a visualization plane generally perpendicular to the longitudinal axis, potentially creating confusion for the clinician. Instead, based on the desired visualization plane (e.g. parallel to the axis of the axis of the passageway or parallel to the wall of the passageway), selected portions of the transducer array 250 may be used to generate an ultrasound image in the desired visualization plane. For example, based on the pose data from the localization sensor 232, which may indicate the orientation of the transducer array 250 relative to a central axis or wall of the anatomic passageway, the transducer elements of the sets 252A and 252C, parallel to the longitudinal axis L, are determined to be in an orientation to generate an image with a visualization plane parallel to the axis L. Ultrasound image data from the selected transducer sets 252A and/or 252C may be used to generate the ultrasound image 262 of the target tissue 113 and the nearby anatomic structures 106, as shown in FIG. 4F. In this example, the ultrasound image 262 may be a sector image in a visualization plane generally parallel to the longitudinal axis L of the passageway 102 (which may also be parallel to the passageway wall 103). The ultrasound image 262 may be in the same or approximately the same visualization plane as the image 260. Generating a consistent point of view, regardless of the orientation of the distal end portion 224, may reduce confusion for the viewing clinician.

Similarly, as shown in FIGS. 4G and 4H, the distal face 235 may have an axial rotation (e.g., roll about a X_{A} axis) approximately forty-five degrees counter-clockwise, relative to the arrangement in FIGS. 4A and 4B. The transducer array 250 and the localization sensor 232 are also rotated approximately forty-five degrees counter-clockwise. Based on the desired visualization plane (e.g. parallel to the axis of the axis of the passageway or parallel to the wall of the passageway), selected portions of the transducer array 250 may be used to generate an ultrasound image in the desired visualization plane. The selected portions of a multi-directional transducer array may be determined based on a various criteria. In some examples, the selected portions of the multi-directional transducer array may be a linear array of transducer elements closest to parallel with the anatomic passageway (e.g., having the smallest angle relative to the longitudinal axis L). In some examples, the selected portions of the multi-directional transducer array may be the consecutive transducer elements of one or more transducer sets that have the smallest distance to the longitudinal axis L, but also span the longest consecutive length (e.g., largest aperture) parallel to the longitudinal axis L. The number of selected transducer elements may be constrained by the number and capacity of the cables that provide electricity and signal handling to the transducer elements. In some examples, a multiplexer device may allow the cables to switch assignments between the various transducer elements. In the example of FIG. 4H, based on the pose data from the localization sensor 232, selected portions 254 of the transducer sets 252A, 252B, 252C, and 252D are determined to be in an orientation to generate an image with a visualization plane parallel to the axis L. In this example, the selected portions 254 may provide the longest aperture available, given the available number of cables. Ultrasound image data from the selected portions 254 may be selected to generate the ultrasound image 264 of the target tissue 113 and the nearby anatomic structures 106, as shown in FIG. 4I. In this example, the ultrasound image 264 may be a sector image in a visualization plane generally parallel to the longitudinal axis L of the passageway 102 (which may also be parallel to the passageway wall 103). The ultrasound image 264 may be in the same or approximately the same visualization plane as the images 260, 262. Generating a consistent point of view, regardless of the orientation of the distal end portion 224, may reduce confusion for the viewing clinician. In other examples, if more cabling is available (e.g., size of the instrument may constrain the number of possible cables), the selected portions may include the entirety of the transducer sets 252A, 252B, 252C, and 252D.

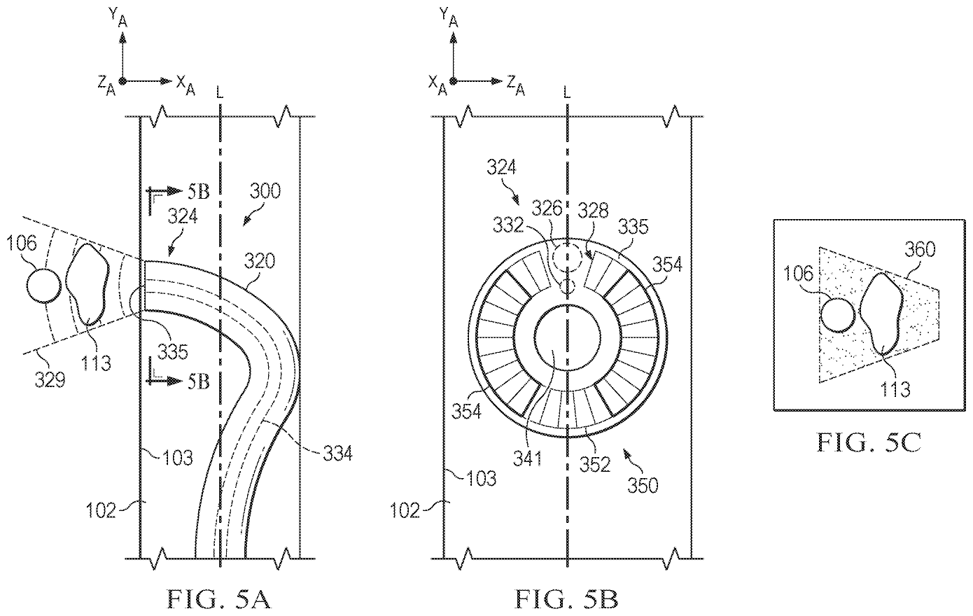

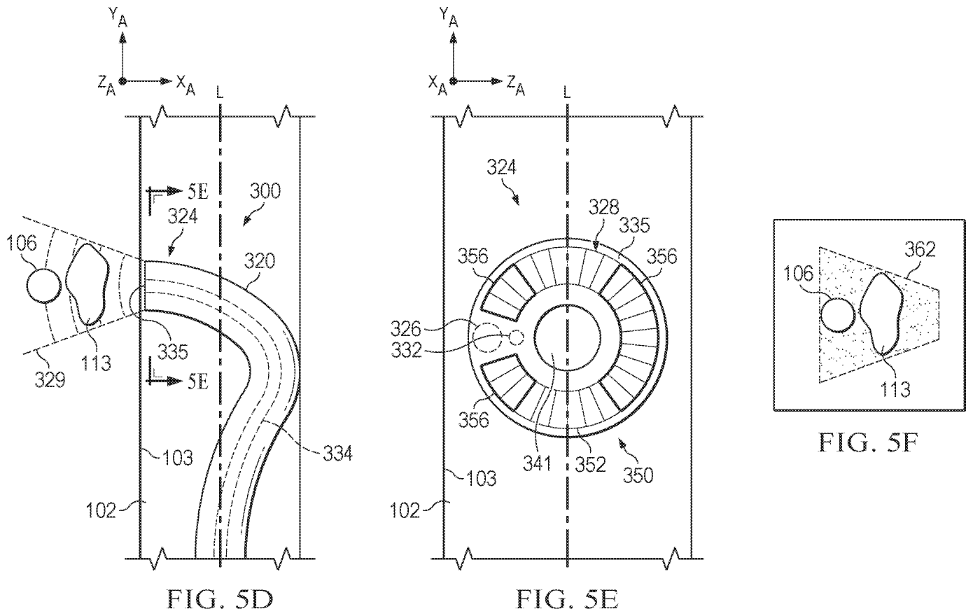

In some examples, a transducer array of a forward-facing imaging device may have a radial or annular arrangement. FIGS. 5A and 5B illustrate a medical instrument system 300, including an elongate flexible instrument 320 which may be similar to the instrument 220, with differences as described. A distal end portion 324 of the elongate flexible instrument 320 may include an imaging device 328, such as an ultrasound imaging device, that has an imaging field of view 329. A channel 334 extending through the elongate flexible instrument 320 may have a distal opening 341 at a distal face 335. In this example, the imaging device 328 may include a forward-facing, multi-directional, ultrasound transducer array 350 including a set 352 of radially arranged transducer elements. In this example, the set 352 may form a ring, partial ring, or a plurality of arc-shaped portions, around the distal opening 341. The forward-facing transducer array 350 may be arranged to image in an antegrade or forward-looking direction. Thus, the imaging field of view 329 of the forward-facing transducer array 350 may extend distally of a distal face 335 of the instrument 320. In this example, a localization sensor 332 may be a shape sensor terminating near the set 352 of radially arranged transducer elements. In other examples, the localization sensor may terminate in other known or predetermined positions and orientations relative to the multi-directional array 350. An optical camera 326 may also generate visible light images from a field of view distal of the distal face 335.

During a procedure, the localization sensor 332 may be registered to the patient anatomy and to a pre-operative model of the anatomic structure 104. With the distal face 335 bent into contact with or close proximity to the passageway wall 103, the transducer array 350 may be aligned generally parallel to the passageway wall 103. Data received from the localization sensor 332 may provide pose information for the distal face 335 and distal end portion 324 of the instrument 320. Because the localization sensor 332 has a known position and orientation relative to the transducer array 350, the orientation of the transducer array 350 may be determined relative to the registered pre-operative model. Based on a desired visualization plane (e.g. parallel to the axis of the axis of the passageway or parallel to the wall of the passageway), selected portions of the transducer array 350 may be used to generate an ultrasound image in the desired visualization plane. For example, selected portions 354 of the transducer set 352 are determined to be in an orientation to generate an image with a visualization plane parallel to the axis L. Ultrasound image data from the selected portions 354 may be selected to generate the ultrasound image 360 of the target tissue 113 and the nearby anatomic structures 106, as shown in FIG. 5C. In this example, the ultrasound image 360 may be a sector image in a visualization plane generally parallel to the longitudinal axis L of the passageway 102 (which may also be parallel to the passageway wall 103).

The bend angle and axial orientation of the instrument 320 may cause the distal end portion 324 to engage the passageway wall 103 at any of a variety of orientations. For example, as shown in FIGS. 5D and 5E, the distal face 335 may have an axial rotation (e.g., roll about a X_{A} axis) approximately ninety degrees counter-clockwise, relative to the arrangement in FIGS. 5A and 5B. Accordingly, the transducer array 550 and the localization sensor 532 are also rotated approximately ninety degrees counter-clockwise. With this rotation, an image generated using the same selected transducer elements as used to generate image 360 of FIG. 5C would be in a visualization plane generally perpendicular to the longitudinal axis, potentially creating confusion for the clinician. Instead, based on the desired visualization plane (e.g. parallel to the axis of the axis of the passageway or parallel to the wall of the passageway), selected portions of the transducer array 350 may be used to generate an ultrasound image in the desired visualization plane. For example, based on the pose data from the localization sensor 332, the selected portion 356 of transducer elements of the set 352 are determined to be in an orientation to generate an image with a visualization plane parallel to the axis L. Ultrasound image data from the selected portion 356 may be selected to generate the ultrasound image 362 of the target tissue 113 and the nearby anatomic structures 106, as shown in FIG. 5F. In this example, the ultrasound image 362 may be a sector image in a visualization plane generally parallel to the longitudinal axis L of the passageway 102 (which may also be parallel to the passageway wall 103). The ultrasound image 362 may be in the same or approximately the same visualization plane as the image 360. Generating a consistent point of view, regardless of the orientation of the distal end portion 324, may reduce confusion for the viewing clinician.

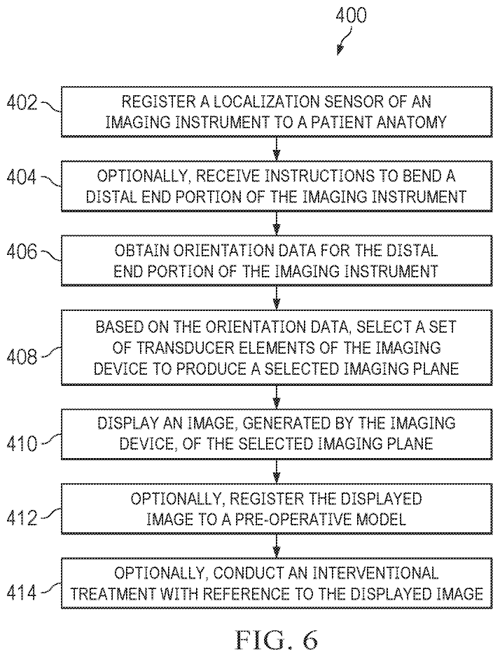

FIG. 6 is a flow chart illustrating a method 400 for generating an image in a selected, predetermined, or otherwise known visualization plane relative to the anatomic passageway. The selected visualization plane may, for example, have an orientation that corresponds to a clinician's expected image plane to minimize confusion, increase efficiency, and improve confidence and precision when sampling or determining characteristics of a target tissue. At a process 402, a localization sensor of an imaging device may be registered to a patient anatomy. The patient anatomy may be further registered to a pre-operative or intra-operative model (e.g., a CT model) of the anatomic structure, including target issue, anatomic passageways, and vulnerable structures. For example, the localization sensor 232 of instrument 220 may be registered to the anatomic structure 104 and, optionally, a pre-operative CT model of the anatomic structure 104.

At an optional process 404, instructions for bending a distal end portion of the imaging instrument may be received. For example, a manipulator of a robot-assisted medical system (e.g. medical system 500) may receive instructions to bend the distal end portion 224 of the instrument 220 so that the distal face 235 of the instrument engages or is positioned near a surface of the anatomic wall 103. With the distal end portion 224 articulated into a bent configuration (e.g., as shown in FIG. 4A, 4D, 4G), the imaging device 228, including the forward-facing, multi-directional, ultrasound transducer array 250 may be aligned generally parallel to the passageway wall 103.

At a process 406, orientation data for the distal end portion of the imaging device may be received. For example, pose data (e.g., including orientation and/or position data) for the distal end portion 224 of the instrument 220 may be obtained from the localization sensor 232. The sensor data may be received, for example, at a control system (e.g., control system 512) of a robot-assisted medical system.

At a process 408, based on the orientation data, a set of transducer elements of the multi-directional imaging array may be selected to produce a selected imaging plane. For example, orientation data from the localization sensor 232, which has a known position and orientation relative to the transducer array 250 and to the registered pre-operative model, may provide information about the orientation of the transducer array 250 relative to the passageway of the registered pre-operative model. Alternatively, without reference to the pre-operative model, shape data from the localization sensor along a region of the instrument proximal of the distal end portion model may provide an indication of the orientation of the axis or wall of the passageway. A desired visualization plane of the transducer array 250 may be selected by a clinician or selected by a control system to provide a consistent frame of reference for viewing the patient anatomy, regardless of the orientation of the distal end portion. In some examples, the desired visualization plane may be an imaging plane parallel to the axis of the axis of the passageway or parallel to the wall of the passageway.

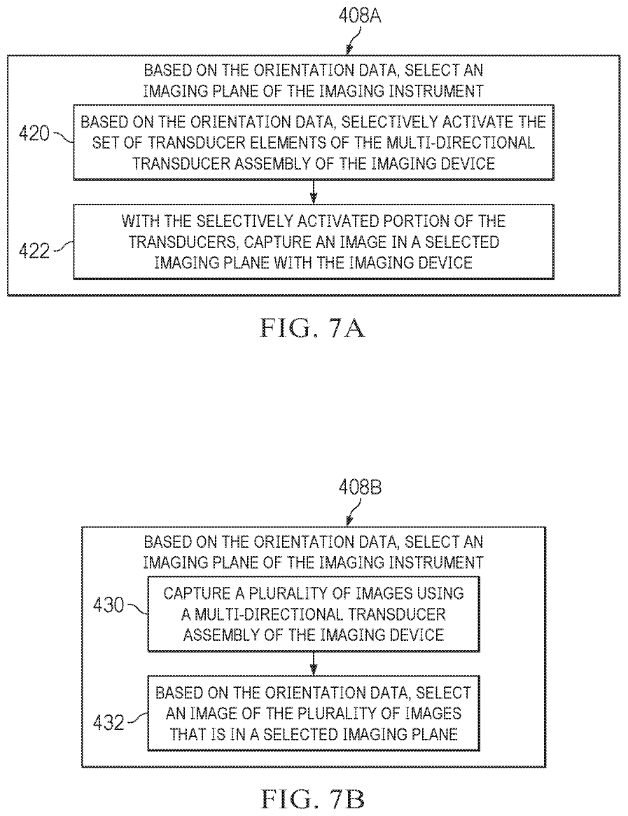

Various techniques for selecting the imaging plane of the imaging instrument are provided by the methods of FIGS. 7A and 7B. FIG. 7A illustrates a method 408A for selecting an imaging plane. At a process 420, based on the orientation data (and optionally, position data) from the localization sensor, the selected set of the transducer elements of a multi-directional transducer assembly of the imaging device may be selectively activated. For example, if the desired visualization plane for a clinician or an image processing system is an imaging plane parallel to the longitudinal axis L of the passageway 102 (which may also be parallel to the passageway wall 103), the orientation information from localization sensor 232 may determine which portion or subset of the transducer elements of transducer array 250 should be activated to generate the image data that will produce an image in the desired plane. A control system (e.g., control system 512) may determine which combination or subsets of transducer elements in the multi-directional imaging array to activate to capture an image in the selected imaging plane. As compared to a full two dimensional array, selectively activating sets or subsets of linearly arranged transducer elements in two dimensions may achieve a longer elevation direction than a fully beamformed two-dimensional array. A longer elevation direction may reduce the number of elements and the associated wiring, minimizing components needed for a small flexible device.

At a process 422, an image in the selected imaging plane may be captured with the selectively activated portion of the transducer elements of the imaging device. For example, with the distal end portion 224 determined by the localization sensor data to be in the orientation as shown in FIG. 4B, the transducer sets 252B and 252D may be selected for activation because they produce an image 260 in the imaging plane parallel to the longitudinal axis L, as shown in FIG. 4C. If the distal end portion 224 is in the orientation as shown in FIG. 4E, the transducer sets 252a and 252C may be selected for activation because they produce an image 262 in the imaging plane parallel to the longitudinal axis L, as shown in FIG. 4F. If the distal end portion 224 is in the orientation as shown in FIG. 4G, the portion 254 of transducer elements may be selected for activation because they produce an image 264 in the imaging plane parallel to the longitudinal axis L, as shown in FIG. 4I. Similarly, orientation data from localization sensor 332 may determine the orientation of the multi-directional transducer assembly 350 which may be used to determine which radially arranged transducer elements to activate to achieve the desired imaging plane as shown in FIG. 5C or 5F.

FIG. 7B illustrates a method 408B for selecting an imaging plane. At a process 430, a plurality of images may be captured using a multi-directional transducer assembly. For example, a plurality of images may be taken in a plurality of image planes with the multi-directional transducer assembly 250. At a process 432, an image may be selected from the plurality of images that corresponds to the selected imaging plane. For example, if the desired visualization plane for a clinician or an image processing system is an imaging plane parallel to the longitudinal axis L of the passageway 102 (which may also be parallel to the passageway wall 103), image data, gathered by the multi-directional transducer assembly 250, that corresponds the imaging plane parallel to the longitudinal axis L may be selected. For example, with the distal end portion 224 determined by the localization sensor data to be in the orientation as shown in FIG. 4B, image data from the transducer sets 252B and 252D may be selected because it will produce an image 260 in the imaging plane parallel to the longitudinal axis L, as shown in FIG. 4C. If the distal end portion 224 is in the orientation as shown in FIG. 4E, image data from the transducer sets 252A and 252C may be selected because it will produce an image 262 in the imaging plane parallel to the longitudinal axis L, as shown in FIG. 4F. If the distal end portion 224 is in the orientation as shown in FIG. 4G, image data from the portion 254 of transducer elements may be selected because it will produce an image 264 in the imaging plane parallel to the longitudinal axis L, as shown in FIG. 4I. Similarly, orientation data from localization sensor 332 may determine the orientation of the multi-directional transducer assembly 350 which may be used to determine from which radially arranged transducer elements image data should be obtained to achieve the desired imaging plane as shown in FIG. 5C or 5F.

In some examples, images from multiple imaging planes may be displayed to a clinician, and the clinician may select which images to view and/or which images to discard. To assist the clinician in deciding, the orientation of the imaging planes may be displayed or otherwise indicated with respect to the longitudinal axis. In some examples, the control system automatically selects the imaging plane from which images will be displayed based on the orientation information obtained while the images were captured. This may reduce confusion and streamline the clinician's workflow.

Referring again to FIG. 6, at a process 410, the image in the selected imaging plane may be displayed on a display system. For example, with the distal end portion arranged as in FIG. 4B, the image 260 may be displayed in the imaging plane parallel to the longitudinal axis L. With the distal end portion arranged as in FIG. 4E, the image 262 may be displayed in the imaging plane parallel to the longitudinal axis L. With the distal end portion arranged as in FIG. 4H, the image 264 may be displayed in the imaging plane parallel to the longitudinal axis L.

At an optional process 412, the displayed image may be registered to a pre-operative anatomic model. For example, the ultrasound image may be co-registered to the anatomic model, and the real-time ultrasound image may be overlayed or concurrently displayed with the pre-operative model. The concurrent display may assist a clinician in performing the interventional procedure. Additionally or alternatively, the co-registered ultrasound image may be used to update the pre-operative model. At an optional process 414, an interventional process, such as a biopsy, may be conducted under the guidance of the displayed image. For example, the interventional tool 236 may be extended from the distal opening 241 to obtain a sample from the target tissue 113. The displayed image may provide an indication of the boundaries of the target tissue and may show the vulnerable tissues 106 that should be avoided by the interventional tool 236. In some examples, the pose to the distal end portion of the instrument may be adjusted to create an improved trajectory for the interventional tool 236 extended from the channel 234. Optionally, any of the processes 402-414 may be repeated for additional interventional procedures.

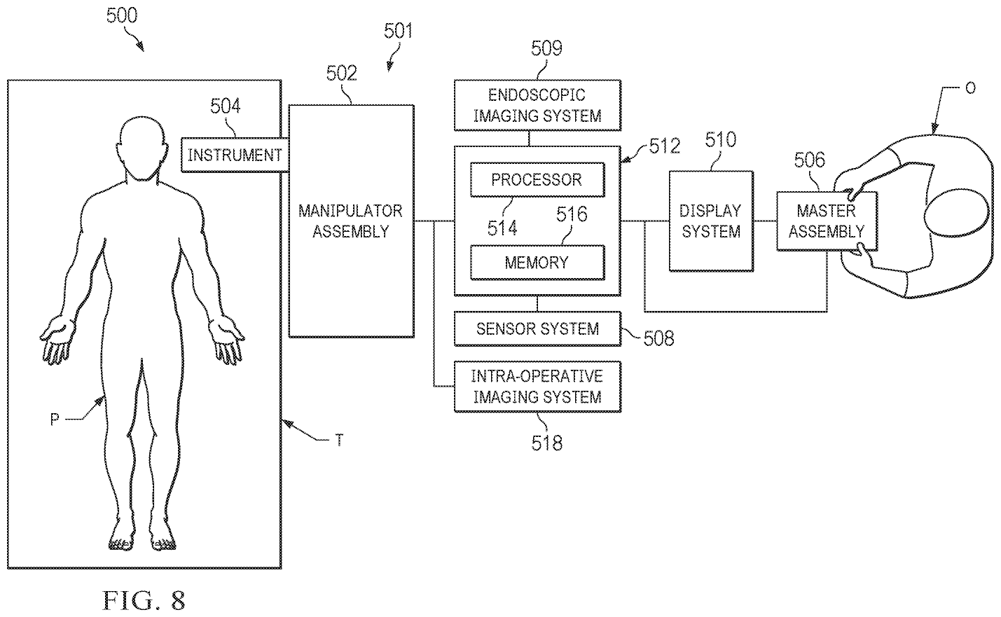

In some examples, the medical procedures described herein may be performed using hand-held or otherwise manually controlled instruments. In other examples, the described instruments and/or tools many be manipulated with a robot-assisted medical system as shown in FIG. 8. FIG. 8 illustrates a robot-assisted medical system 500. The robot-assisted medical system 500 generally includes a manipulator assembly 502 for operating a medical instrument system 504 (including, for example, medical instrument system 100, 120, 200, 300) in performing various procedures on a patient P positioned on a table T in a surgical environment 501. The manipulator assembly 502 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 506, which may be inside or outside of the surgical environment 501, generally includes one or more control devices for controlling manipulator assembly 502. Manipulator assembly 502 supports medical instrument system 504 and may include a plurality of actuators or motors that drive inputs on medical instrument system 504 in response to commands from a control system 512. The actuators may include drive systems that when coupled to medical instrument system 504 may advance medical instrument system 1104 into a naturally or surgically created anatomic orifice. Other drive systems may move the distal end of medical instrument system 504 in multiple degrees of freedom, which may include three degrees of linear motion (e.g., linear motion along the X, Y, Z Cartesian axes) and/or three degrees of rotational motion (e.g., rotation about the X, Y, Z Cartesian axes). Additionally, the actuators can be used to actuate an articulatable end effector of medical instrument system 504 for grasping tissue in the jaws of a biopsy device and/or the like.

Robot-assisted medical system 500 also includes a display system 510 (which may display, for example, an ultrasound image generated by imaging devices and systems described herein) for displaying an image or representation of the interventional site and medical instrument system 504 generated by a sensor system 508 (including, for example, and ultrasound sensor) and/or an endoscopic imaging system 509. Display system 510 and master assembly 606 may be oriented so operator O can control medical instrument system 504 and master assembly 606 with the perception of telepresence.

In some examples, medical instrument system 504 may include components for use in surgery, biopsy, ablation, illumination, irrigation, or suction. In some examples, medical instrument system 504 may include components of the endoscopic imaging system 509, which may include an imaging scope assembly or imaging instrument (e.g. a visible light and/or near infrared light imaging) that records a concurrent or real-time image of a interventional site and provides the image to the operator or operator O through the display system 510. The concurrent image may be, for example, a two or three-dimensional image captured by an imaging instrument positioned within the interventional site. In some examples, the endoscopic imaging system components may be integrally or removably coupled to medical instrument system 504. However, in some examples, a separate endoscope, attached to a separate manipulator assembly may be used with medical instrument system 504 to image the interventional site. The endoscopic imaging system 509 may be implemented as hardware, firmware, software, or a combination thereof which interact with or are otherwise executed by one or more computer processors, which may include the processors of the control system 512.

The sensor system 508 may include a position/location sensor system (e.g., an electromagnetic (EM) sensor system) and/or a shape sensor system for determining the position, orientation, speed, velocity, pose, and/or shape of the medical instrument system 504.

Robot-assisted medical system 500 may also include control system 512. Control system 512 includes at least one memory 516 and at least one computer processor 514 for effecting control between medical instrument system 504, master assembly 506, sensor system 508, endoscopic imaging system 509, intra-operative imaging system 518, and display system 510. Control system 512 also includes programmed instructions (e.g., a non-transitory machine-readable medium storing the instructions) to implement some or all of the methods described in accordance with aspects disclosed herein, including instructions for providing information to display system 510.

Control system 512 may further include a virtual visualization system to provide navigation assistance to operator O when controlling medical instrument system 504 during an image-guided interventional procedure. Virtual navigation using the virtual visualization system may be based upon reference to an acquired pre-operative or intra-operative dataset of anatomic passageways. The virtual visualization system processes images of the interventional site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intra-operative imaging system 518 may be arranged in the surgical environment 501 near the patient P to obtain images of the anatomy of the patient P during a medical procedure. The intra-operative imaging system 518 may provide real-time or near real-time images of the patient P. In some examples, the intra-operative imaging system 518 may comprise an ultrasound imaging system for generating two-dimensional and/or three-dimensional images. For example, the intra-operative imaging system 518 may be at least partially incorporated into sensing instrument 200. In this regard, the intra-operative imaging system 518 may be partially or fully incorporated into the medical instrument system 504.

FIG. 9A is a simplified diagram of a medical instrument system 600 configured in accordance with various embodiments of the present technology. The medical instrument system 600 includes an elongate flexible device 602 (e.g., delivery catheter 240), such as a flexible catheter, coupled to a drive unit 604. The elongate flexible device 602 includes a flexible body 616 having a proximal end 617 and a distal end or tip portion 618. The medical instrument system 600 further includes a tracking system 630 for determining the position, orientation, speed, velocity, pose, and/or shape of the distal end 918 and/or of one or more segments 624 along the flexible body 616 using one or more sensors and/or imaging devices as described in further detail below.

The tracking system 630 may optionally track the distal end 618 and/or one or more of the segments 624 using a shape sensor 622. The shape sensor 622 may optionally include an optical fiber aligned with the flexible body 616 (e.g., provided within an interior channel (not shown) or mounted externally). The optical fiber of the shape sensor 622 forms a fiber optic bend sensor for determining the shape of the flexible body 616. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. No. 7,781,724 (filed Sep. 26, 2006, disclosing “Fiber optic position and shape sensing device and method relating thereto”; U.S. Pat. No. 7,772,541, filed Mar. 12, 2008, titled “Fiber Optic Position and/or Shape Sensing Based on Rayleigh Scatter”; and U.S. Pat. No. 6,389,187, filed Apr. 21, 2000, disclosing “Optical Fiber Bend Sensor,” which are all incorporated by reference herein in their entireties. In some embodiments, the tracking system 630 may optionally and/or additionally track the distal end 618 using a position sensor system 620. The position sensor system 620 may be a component of an EM sensor system with the position sensor system 620 including one or more conductive coils that may be subjected to an externally generated electromagnetic field. In some embodiments, the position sensor system 620 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, filed Aug. 9, 1999, disclosing “Six-Degree of Freedom Tracking System Having a Passive Transponder on the Object Being Tracked,” which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g., fiber shape sensors, EM sensors, and/or the like) may be integrated within the medical instrument 626 and used to track the position, orientation, speed, velocity, pose, and/or shape of a distal end or portion of medical instrument 626 using the tracking system 630.

The flexible body 616 includes a channel 621 sized and shaped to receive a medical instrument 626 (e.g., instrument 120, 200, 300). FIG. 9B, for example, is a simplified diagram of the flexible body 616 with the medical instrument 626 extended according to some embodiments. In some embodiments, the medical instrument 626 may be used for procedures such as imaging, visualization, surgery, biopsy, ablation, illumination, irrigation, and/or suction. The medical instrument 626 can be deployed through the channel 621 of the flexible body 616 and used at a target location within the anatomy. The medical instrument 626 may include, for example, image capture probes, biopsy instruments, ablation needles, electroporation needles, laser ablation fibers, and/or other surgical, diagnostic, or therapeutic tools, including any of the instrument systems described above. The medical instrument 626 may be advanced from the opening of channel 621 to perform the procedure and then be retracted back into the channel 621 when the procedure is complete. The medical instrument 626 may be removed from the proximal end 617 of the flexible body 616 or from another optional instrument port (not shown) along the flexible body 616.

In some examples, an optical or visible light imaging instrument (e.g., an image capture probe) may extend within the channel 621 or within the structure of the flexible body 616. The imaging instrument may include a cable coupled to the camera for transmitting the captured image data. In some embodiments, the imaging instrument may be a fiber-optic bundle, such as a fiberscope, that couples to an image processing system 631. The imaging instrument may be single or multi-spectral, for example capturing image data in one or more of the visible, infrared, and/or ultraviolet spectrums.

The flexible body 616 may also house cables, linkages, or other steering controls (not shown) that extend between the drive unit 604 and the distal end 9618 to controllably bend the distal end 618 as shown, for example, by broken dashed line depictions 619 of the distal end 618. In some embodiments, at least four cables are used to provide independent “up-down” steering to control a pitch of the distal end 618 and “left-right” steering to control a yaw of the distal end 618. Steerable elongate flexible devices are described in detail in U.S. Pat. No. 9,452,276, filed Oct. 14, 2011, disclosing “Catheter with Removable Vision Probe,” and which is incorporated by reference herein in its entirety. In various embodiments, medical instrument 626 may be coupled to drive unit 604 or a separate second drive unit (not shown) and be controllably or robotically bendable using steering controls.

The information from the tracking system 630 may be sent to a navigation system 632 where it is combined with information from the image processing system 631 and/or the preoperatively obtained models to provide the operator with real-time position information. In some embodiments, the real-time position information may be displayed on the display system 510 of FIG. 8 for use in the control of the medical instrument system 600. In some embodiments, the control system 512 of FIG. 8 may utilize the position information as feedback for positioning the medical instrument system 600. Various systems for using fiber optic sensors to register and display a surgical instrument with surgical images are provided in U.S. Pat. No. 8,900,131, filed May 13, 2011, disclosing “Medical System Providing Dynamic Registration of a Model of an Anatomic Structure for Image-Guided Surgery,” which is incorporated by reference herein in its entirety.

In some embodiments, the medical instrument system 600 may be teleoperated or robot-assisted within the medical system 500 of FIG. 8. In some embodiments, the manipulator assembly 502 of FIG. 8 may be replaced by direct operator control. In some embodiments, the direct operator control may include various handles and operator interfaces for hand-held operation of the instrument.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 1112) or may be implemented, at least in part, in the form of executable code stored on non-transitory, tangible, machine-readable media that when run by one or more processors (e.g., the processors 1114 of control system 1112) may cause the one or more processors to perform one or more of the processes.

One or more elements in examples of this disclosure may be implemented in software to execute on a processor of a computer system such as control processing system. When implemented in software, the elements of the examples may be the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples described herein are not described with reference to any particular programming language. It will be appreciated that a variety of programming languages may be used to implement the teachings described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

While certain illustrative examples have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

1. A system comprising:
an elongate flexible instrument including
an imaging device disposed at a distal end portion of the elongate flexible instrument, the imaging device including a multi-directional imaging array and
a localization sensor within the elongate flexible instrument; and
a controller comprising one or more processors configured to:
register the localization sensor to a patient anatomy;
receive orientation data for the distal end portion of the elongate flexible instrument from the localization sensor;
based on the orientation data, select a set of transducer elements of the multi-directional imaging array to produce a selected imaging plane; and
display an image of the selected imaging plane, the image generated by imaging data from the multi-directional imaging array of the imaging device.

2. The system of claim 1, wherein the imaging device includes an ultrasound imaging device.

3. The system of claim 1, wherein the localization sensor includes an optical fiber shape sensor.

4. The system of claim 1, wherein the localization sensor includes an electromagnetic sensor.

5. The system of claim 1, wherein the multi-directional imaging array includes a forward-facing imaging array.

6. The system of claim 1, wherein the multi-directional imaging array includes a first linear transducer set and a second linear transducer set extending orthogonally to the first linear transducer set.

7. The system of claim 1, wherein the multi-directional imaging array includes a radial transducer array.

8. The system of claim 1, wherein selecting the set of transducer elements of the multi-directional imaging array to produce the selected imaging plane includes:
selectively activating s the set of transducer elements of the multi-directional imaging array, based on the orientation data and
generating the imaging data in the selected imaging plane with the selectively activated set of transducer elements of the multi-directional imaging array.

9. The system of claim 8, wherein the selectively activated set of transducer elements of the multi-directional imaging array includes a first subset of transducer elements and a second subset of transducer elements, wherein the first and second subsets of transducer elements are separated from each other on opposite sides of a channel opening on a distal face of the elongate flexible instrument.

10. The system of claim 1, wherein the one or more processors are further configured to:
capture a plurality of images with the multi-directional imaging array and
select an image of the plurality of images that is in the selected imaging plane, based on the orientation data.

11. The system of claim 1, wherein the selected imaging plane has a parallel orientation to a longitudinal axis of an anatomic passageway in which the elongate flexible instrument extends.

12. The system of claim 1, wherein the selected imaging plane has a parallel orientation to a longitudinal axis of a portion of the elongate flexible instrument proximal of the distal end portion.

13. The system of claim 1, wherein the one or more processors are further configured to adjust a pose of the distal end portion of the elongate flexible instrument prior to conducting an interventional treatment with an interventional tool extended through an aperture in a distal face of the elongate flexible instrument.

14. The system of claim 1, wherein the one or more processors are further configured to register the image in the selected imaging plane to a pre-operative anatomic model.

15. The system of claim 1, wherein the one or more processors are further configured to receive pose data for the distal end portion of the elongate flexible instrument from the localization sensor, the pose data including the orientation data.

16. The system of claim 1, wherein the one or more processors are further configured to display at least one guidance marker to guide motion of the distal end portion of the elongate flexible instrument into an apposition position.

17. The system of claim 16, wherein the at least one guidance marker includes an extension marker for guiding an extension of the distal end portion and an apposition marker for guiding bending the distal end portion into the apposition position.

18. A method comprising:
registering a localization sensor to a patient anatomy, the localization sensor extending within an elongate flexible instrument;
receiving orientation data for a distal end portion of the elongate flexible instrument from the localization sensor;
based on the orientation data, selecting a set of transducer elements of a multi-directional imaging array of an imaging device disposed at a distal end of the elongate flexible instrument to produce a selected imaging plane; and
displaying an image of the selected imaging plane, the image generated by imaging data from the multi-directional imaging array of the imaging device.

19. The method of claim 18, further comprising receiving ultrasound imaging data from the multi-directional imaging array.

20-21. (canceled)

22. The method of claim 18, wherein the localization sensor includes an optical fiber shape sensor.

23.-32 (canceled)
